(19) United States
(12) Patent Application Publication
(10) Pub. No.: US 2021/0368839 A1
(43) Pub. Date: Dec. 2, 2021

(54) GLUCOSYL STEVIA COMPOSITIONS

(71) Applicant: PureCircle USA Inc., Chicago, IL (US)

(72) Inventor: Avetik MARKOSYAN, Yerevan (AM)

(21) Appl. No.: 16/467,805

(22) PCT Filed: Dec. 8, 2017
(86) PCT No.: PCT/US17/65235

§ 371 Date: Jun. 7, 2019

Related U.S. Application Data
(63) Continuation-in-part of application No. 15/646,629, filed on Jul. 11, 2017, now abandoned.
Application No. 15/646,629 is a continuation-in-part of application No. 15/470,388, filed on Mar. 27, 2017, now abandoned.
Application No. 15/470,388 is a continuation-in-part of application No. 14/273,056, filed on May 8, 2014, now Pat. No. 10,531,683.
Application No. 14/273,056 is a division of application No. 13/016,545, filed on Jan. 28, 2011, now Pat. No. 8,790,730.
Application No. 13/016,545 is a continuation-in-part of application No. 12/720,888, filed on Mar. 10, 2010, now Pat. No. 8,334,006.
Application No. 12/720,888 is a continuation-in-part of application No. 11/246,066, filed on Oct. 11, 2005, now Pat. No. 7,807,206.
Application No. 11/246,066 is a continuation-in-part of application No. 11/246,152, filed on Oct. 11, 2005, now Pat. No. 7,862,845.
Application No. 11/246,152 is a continuation-in-part of application No. 12/684,129, filed on Jan. 8, 2010, now Pat. No. 8,318,232.
Application No. 12/684,129 is a continuation-in-part of application No. 12/684,130, filed on Jan. 8, 2010, now Pat. No. 8,323,716.
Application No. 12/684,130 is a continuation-in-part of application No. 12/684,981, filed on Jan. 11, 2010, now Pat. No. 8,298,599.
Application No. PCT/US17/65235 is a continuation-in-part of application No. 15/437,208, filed on Feb. 20, 2017, now Pat. No. 10,398,160.
Application No. 15/437,208 is a division of application No. 14/742,457, filed on Jun. 17, 2015, now Pat. No. 9,585,420.
Application No. 14/742,457 is a division of application No. 14/031,290, filed on Sep. 19, 2013, now Pat. No. 9,107,436.
Application No. 14/031,290 is a continuation-in-part of application No. 14/005,852, filed on Sep. 18, 2013, now abandoned.
Application No. 14/031,290 is a continuation-in-part of application No. 14/005,850, filed on Sep. 18, 2013, now abandoned.
Application No. 14/031,290 is a continuation-in-part of application No. 13/567,707, filed on Aug. 6, 2012, now Pat. No. 8,647,844.
Application No. 13/567,707 is a division of application No. 13/029,263, filed on Feb. 17, 2011, now Pat. No. 8,257,948.
Application No. 14/031,290 is a continuation-in-part of application No. 13/589,754, filed on Aug. 20, 2012, now Pat. No. 8,735,101.
Application No. 13/589,754 is a continuation of application No. 13/074,179, filed on Mar. 29, 2011, now Pat. No. 8,318,459.
Application No. 14/031,290 is a continuation-in-part of application No. 13/841,261, filed on Mar. 15, 2013, now abandoned.
Application No. PCT/US17/65235 is a continuation-in-part of application No. 15/550,075, filed on Aug. 10, 2017.
Application No. 15/550,075 is a continuation-in-part of application No. 14/254,653, filed on Apr. 16, 2014, now Pat. No. 9,386,797.
Application No. PCT/US17/65235 is a continuation-in-part of application No. 15/213,013, filed on Jul. 18, 2016, now Pat. No. 10,729,163.
Application No. 15/213,013 is a continuation of application No. 14/254,627, filed on Apr. 16, 2014, now Pat. No. 9,392,799.
Application No. 14/254,627 is a continuation of application No. 13/589,754, filed on Aug. 20, 2012, now Pat. No. 8,735,101.
Application No. 13/589,754 is a continuation of application No. 13/074,179, filed on Mar. 29, 2011, now Pat. No. 8,318,459.
Application No. 13/074,179 is a continuation-in-part of application No. 13/029,263, filed on Feb. 17, 2011, now Pat. No. 8,257,948.
Application No. PCT/US17/65235 is a continuation-in-part of application No. 15/243,504, filed on Aug. 22, 2016, now Pat. No. 10,130,116.
Application No. 15/243,504 is a division of application No. 14/795,792, filed on Jul. 9, 2015, now Pat. No. 9,420,815.
Application No. 14/795,792 is a continuation of application No. 14/031,290, filed on Sep. 19, 2013, now Pat. No. 9,107,436.
Application No. 14/031,290 is a continuation-in-part of application No. 14/005,852, filed on Sep. 18, 2013, now abandoned.
Application No. 14/005,852 is a continuation-in-part of application No. 14/005,850, filed on Sep. 18, 2013, now abandoned.
Application No. 14/005,850 is a continuation-in-part of application No. 13/567,707, filed on Aug. 6, 2012, now Pat. No. 8,647,844.
Application No. 13/567,707 is a division of application No. 13/029,263, filed on Feb. 17, 2011, now Pat. No. 8,257,948.
Application No. 14/031,290 is a continuation-in-part of application No. 13/589,754, filed on Aug. 20, 2012, now Pat. No. 8,735,101.
Application No. 13/589,754 is a continuation of application No. 13/074,179, filed on Mar. 29, 2011, now Pat. No. 8,318,459.
Application No. 14/031,290 is a continuation-in-part of application No. 13/841,261, filed on Mar. 15, 2013, now abandoned.
Application No. PCT/US17/65235 is a continuation-in-part of application No. 15/332,760, filed on Oct. 24, 2016.
Application No. 15/332,760 is a division of application No. 14/352,832, filed on Aug. 18, 2014, now Pat. No. 9,474,296.
Application No. 14/352,832 is a continuation-in-part of application No. 13/567,707, filed on Aug. 6, 2012, now Pat. No. 8,647,844.
Application No. 13/567,707 is a division of application No. 13/029,263, filed on Feb. 17, 2011, now Pat. No. 8,257,948.
(60) Provisional application No. 62/432,172, filed on Dec. 9, 2016.
(60) Provisional application No. 61/260,593, filed on Nov. 12, 2009.
(60) Provisional application No. 61/290,778, filed on Dec. 29, 2009.
(60) Provisional application No. 61/725,233, filed on Nov. 12, 2012.
(60) Provisional application No. 62/114,134, filed on Feb. 10, 2015.
(60) Provisional application No. 61/725,233, filed on Nov. 12, 2012.
(60) Provisional application No. 61/580,274, filed on Dec. 26, 2011.
(60) Provisional application No. 61/548,818, filed on Oct. 19, 2011.

(51) Int. Cl. A23L 27/30; A23G 3/48; A23G 9/42; A21D 13/062; A23G 9/32; A21D 2/18; A23L 2/60; A23C 9/13; A23G 3/36; A21D 2/36; A23L 27/50

(52) U.S. Cl. CPC A23L 27/36; A23G 3/48; A23G 9/42; A21D 13/062; A23G 9/32; A21D 2/181; A23V 2002/00; A23C 9/1307; A23G 3/36; A21D 2/36; A23L 27/33; A23L 27/34; A23L 27/50; A23L 2/60

(57) ABSTRACT

Glucosyl steviol glycosides (GSG) compositions are prepared from steviol glycosides of Stevia rebaudiana. The GSG compositions can be used as sweeteners in various consumables including foods, beverages, cosmetics and pharmaceuticals.

### RELATED APPLICATIONS

This patent application claims priority to U.S. Provisional Patent Application No. 62/432,172, filed on Dec. 9, 2016, which is incorporated by reference herein in its entirety.

For purposes of the United States of America, this patent application is also a continuation-in-part of the following applications, each of which is incorporated by reference herein in its entirety: U.S. patent application Ser. No. 15/646,629, filed on Jul. 11, 2017; U.S. patent application Ser. No. 15/470,388, filed on Mar. 27, 2017; U.S. patent application Ser. No. 14/273,056, filed on May 8, 2014; U.S. patent application Ser. No. 15/437,208, filed on Feb. 20, 2017; U.S. patent application Ser. No. 15/550,075, filed on Aug. 10, 2017; U.S. patent application Ser. No. 15/213,013, filed on Jul. 18, 2016; U.S. patent application Ser. No. 15/243,504, filed on Aug. 22, 2016; U.S. patent application Ser. No. 15/332,760, filed on Oct. 24, 2016; and U.S. patent application Ser. No. 14/494,322, filed on Sep. 23, 2014.

This patent application also incorporates by reference each of the following patents in its entirety: U.S. Pat. Nos. 7,807,206, 7,838,044, 8,257,948, 8,318,459, 8,647,844, 8,669,077, 8,318,232, 8,323,716, 8,735,101, 8,911,971, 8,993,269, 9,055,761, 9,107,436, 9,386,797, 9,392,799, 9,420,815, 9,474,296, 9,585,420, 9,603,373, and 9,706,792. This patent application also incorporates each of the following patent applications in its entirety: U.S. patent application Ser. No. 14/005,852, published as US 2014/0023750; U.S. patent application Ser. No. 14/005,850, published as US 2014/0010917; and U.S. patent application Ser. No. 14/494,322, published as US 2015/0125571.

### FIELD OF THE INVENTION

The invention relates to a process for producing a sweetener from the extract of the Stevia rebaudiana plant and its use in various food products and beverages.

### DESCRIPTION OF THE RELATED ART

Nowadays sugar alternatives are receiving increasing attention due to awareness of many diseases in conjunction with consumption of high-sugar foods and beverages. However many artificial sweeteners such as dulcin, sodium cyclamate and saccharin were banned or restricted in some countries due to concerns on their safety. Therefore non-caloric sweeteners of natural origin are becoming increasingly popular. The sweet herb Stevia rebaudiana Bertoni, produces a number of diterpene glycosides which feature high intensity sweetness and sensory properties superior to those of many other high potency sweeteners.

The above-mentioned sweet glycosides, have a common aglycon, steviol, and differ by the number and type of carbohydrate residues at the C13 and C19 positions. The leaves of Stevia are able to accumulate up to 10-20% (on dry weight basis) steviol glycosides. The major glycosides found in Stevia leaves are Rebaudioside A (2-10%), Stevioside (2-10%), and Rebaudioside C (1-2%). Other glycosides such as Rebaudioside B, D, E, and F, Steviolbioside and Rubusoside are found at much lower levels (approx. 0-0.2%).

Two major glycosides—Stevioside and Rebaudioside A, were extensively studied and characterized in terms of their suitability as commercial high intensity sweeteners. Stability studies in carbonated beverages confirmed their heat and pH stability (Chang S. S., Cook, J. M. (1983) Stability studies of stevioside and Rebaudioside A in carbonated beverages. J. Agric. Food Chem. 31: 409-412.)

Steviol glycosides differ from each other not only by molecular structure, but also by their taste properties. Usually stevioside is found to be 110-270 times sweeter than sucrose, Rebaudioside A between 150 and 320 times, and Rebaudioside C between 40-60 times sweeter than sucrose. Dulcoside A is 30 times sweeter than sucrose. Rebaudioside A has the least astringent, the least bitter, and the least persistent aftertaste thus possessing the most favorable sensory attributes in major steviol glycosides (Tanaka O. (1987) Improvement of taste of natural sweetners. Pure Appl. Chem. 69:675-683; Phillips K. C. (1989) Stevia: steps in developing a new sweetener. In: Grenby T. H. ed. Developments in sweeteners, vol. 3. Elsevier Applied Science, London. 1-43.)

Methods for the extraction and purification of sweet glycosides from the Stevia rebaudiana plant using water or organic solvents are described in, for example, U.S. Pat. Nos. 4,361,697; 4,082,858; 4,892,938; 5,972,120; 5,962,678; 7,838,044 and 7,862,845.

However, even in a highly purified state, steviol glycosides still possess undesirable taste attributes such as bitterness, sweet aftertaste, licorice flavor, etc. One of the main obstacles for the successful commercialization of stevia sweeteners are these undesirable taste attributes. It was shown that these flavor notes become more prominent as the concentration of steviol glycosides increases (Prakash I., DuBois G. E., Clos J. F., Wilkens K. L., Fosdick L. E. (2008) Development of rebiana, a natural, non-caloric sweetener. Food Chem. Toxicol., 46, S75-S82.)

On the other hand, replacing large amounts of sugar in the formulations brings up such problems as reduced mouthfeel, incomplete flavor profile etc. Therefore the application of high intensity low calorie sweeteners has to provide solutions to address these problems.

Thus, if a single composition will be able to deliver not only sweetness, but also possess flavor enhancing properties and correct the incomplete mouthfeel associated with the elimination of sucrose from food and beverage formulations, it will certainly be advantageous compared to other high intensity sweeteners known in the art.

Therefore it is necessary to develop new generation of stevia sweeteners which will possess improved organoleptic properties.

### SUMMARY OF THE INVENTION

The present invention is aimed to overcome the disadvantages of existing steviol glycosides (SG) sweeteners. The invention describes a process for producing a high quality food ingredient from the extract of the Stevia rebaudiana plant and use thereof in various consumables, including food products and beverages, as sweetener.

The invention, in part, pertains to an ingredient comprising glucosylated derivatives of steviol glycosides of Stevia rebaudiana plant. The steviol glycosides are selected from the group consisting of stevioside, Rebaudioside A, Rebaudioside B, Rebaudioside C, Rebaudioside D, Rebaudioside E, Rebaudioside F, Rebaudioside G, Rebaudioside H, Rebaudioside I, Rebaudioside J, Rebaudioside K, Rebaudioside L, Rebaudioside M, Rebaudioside N, Rebaudioside O, Rebaudioside Q, dulcoside A, steviolbioside, rubusoside, as well as other steviol glycosides found in Stevia rebaudiana plant and combinations thereof.

The invention, in part, pertains to a process for producing an ingredient comprising glucosylated forms of stevioside, Rebaudioside A, Rebaudioside B, Rebaudioside C, Rebaudioside D, Rebaudioside E, Rebaudioside F, Rebaudioside G, Rebaudioside H, Rebaudioside I, Rebaudioside J, Rebaudioside K, Rebaudioside L, Rebaudioside M, Rebaudioside N, Rebaudioside O, Rebaudioside Q, dulcoside A, steviolbioside, rubusoside, as well as other steviol glycosides found in Stevia rebaudiana plant and combinations thereof.

In one embodiment the process includes enzymatic transglucosylation step using CGTase.

In another embodiment the process includes enzymatic transglucosylation step using CGTase produced by cultures of Bacillus stearothermophilus.

The process may include the step of shortening glucosyl chains by β-amylase.

The process may also include at least one step selected from the group including decolorizing, desalting, removing unreacted dextrins, concentrating, drying and combinations thereof.

The decolorizing can be performed using activated carbon, ion-exchange resins, membrane filters, or any other method known to art for decolorizing solutions comprising organic molecules. The desalting can be performed by passing through ion-exchange resins, membrane filters any other method known to art for desalting solutions comprising organic molecules. Removing the unreacted dextrins can be performed by passing through macroporuos polymeric resin. Concentrating can be performed by evaporators, membrane filters any other method known to art for concentrating solutions comprising organic molecules. Drying may be performed by spray drier, flash drier, vacuum dryer, tray dryer, freeze dryer or any other method known to art for drying solutions comprising organic molecules.

In one embodiment, Stevia extract commercialized by PureCircle (JiangXi) Co., Ltd. (China), containing stevioside (28-30%), Rebaudioside A (50-55%), Rebaudioside C (9-12%), Rebaudioside F (1-3%) and other glycosides was used as a starting material. Alternatively stevia extracts with different ratio of steviol glycosides as well as highly purified steviol glycosides such as Rebaudioside A, Rebaudioside B, Rebaudioside C, Rebaudioside D, Rebaudioside E, Rebaudioside F, Rebaudioside G, Rebaudioside H, Rebaudioside I, Rebaudioside J, Rebaudioside K, Rebaudioside L, Rebaudioside M, Rebaudioside N, Rebaudioside O, Rebaudioside Q, dulcoside A, steviolbioside, rubusoside, as well as other steviol glycosides found in Stevia rebaudiana plant, may be used as starting materials.

In one embodiment the starting material steviol glycosides were obtained via fermentation of recombinant microorganism.

In another embodiment the starting material steviol glycosides were obtained via biotransformation of steviol glycosides extracted from Stevia plant.

In yet another embodiment the starting material steviol glycosides were obtained via chemical synthesis.

The starting material was subjected to the enzymatic transglucosylation by action of cyclodextrin glycosyltransferase (CGTase) in the presence of starch as a glucose donor. As a result α-1,4-glucosyl derivatives were formed with different degree of polymerization. Alternatively other glucose donors selected from the group including but not limited to maltodextrins, hydrolyzed starch, maltooligosaccharides, corn syrup solids, cyclodextrins, may be used.

In one embodiment, the unreacted dextrins from obtained reaction mixture were removed by macroporous adsorption resin.

In yet another embodiment the reaction mixture was decolorized.

In one embodiment the reaction mixture was desalted.

In another embodiment the reaction mixture was concentrated.

In yet another embodiment the reaction mixture was spray dried.

The obtained products were applied in various consumables including foods and beverages as sweeteners.

It is to be understood that both the foregoing general description and the following detailed description are exemplary and explanatory and are intended to provide further explanation of the invention as claimed.

### BRIEF DESCRIPTION OF THE DRAWINGS

The accompanying drawings are included to provide a further understanding of the invention. The drawings illustrate embodiments of the invention and together with the description serve to explain the principles of the embodiments of the invention.

FIG. 1 shows an exemplary HPLC chromatogram of glucosylated steviol glycosides (GSG).

### DETAILED DESCRIPTION OF THE INVENTION

Advantages of the present invention will become more apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

In one embodiment, Stevia extract commercialized by PureCircle (JiangXi) Co., Ltd. (China), containing stevioside (28-30%), Rebaudioside A (50-55%), Rebaudioside C (9-12%), Rebaudioside F (1-3%) and other glycosides (hereinafter collectively, “steviol glycosides”) amounting to total steviol glycosides content of at least 95%, was used as a starting material. Alternatively stevia extracts with different ratio of steviol glycosides as well as highly purified steviol glycosides such as Rebaudioside A, Rebaudioside B, Rebaudioside C, Rebaudioside D, Rebaudioside E, Rebaudioside F, Rebaudioside G, Rebaudioside H, Rebaudioside I, Rebaudioside J, Rebaudioside K, Rebaudioside L, Rebaudioside M, Rebaudioside N, Rebaudioside O, Rebaudioside Q, dulcoside A, steviolbioside, rubusoside, as well as other steviol glycosides found in Stevia rebaudiana plant, may be used as starting materials.

The HPLC analysis of the raw materials and products was performed on Agilent Technologies 1200 Series (USA) liquid chromarograph, equipped with Zorbax-NH₂ (4.6×250 mm) column. The mobile phase was acetonitrile-water gradient from 80:20, v/v (0-2 min) to 50:50, v/v (2-70 min). A diode array detector set at 210 nm was used as the detector.

The transglucosylation was accomplished by cyclomaltodextrin glucanotransferases (CGTases; EC 2.4.1.19) produced by Bacillus stearothermophilus St-88 (PureCircle Sdn Bhd Collection of Industrial Microorganisms—Malaysia). However, any other CGTase or enzyme possessing intermolecular transglucosylation activity may be applied as well. The enzyme can be in a form of cell-free culture broth, concentrated liquid cell-free culture broth, spray dried or freeze dried cell-free culture broth, or high purity protein. Free and immobilized enzyme preparations can be used.

The activity of CGTase preparations was determined according to the procedure described in Hale W. S., Rawlins L. C. (1951) Amylase of Bacillus macerans. Cereal Chem. 28, 49-58.

Starches of different origin may be used as donors of glucosyl units such as, derived from wheat, corn, potato, tapioca, and sago. Alternatively other glucose donors selected from the group including but not limited to maltodextrins, hydrolyzed starch, cyclodextrins, corn syrup solids may be used.

In one embodiment the starch was subjected to partial hydrolysis (liquefaction) prior to the transglycosylation reaction. The dextrose equivalent of the partially hydrolyzed starch can be in the range of about 10-25, preferably about 12-16. Any enzyme capable of starch hydrolysis may be used for liquefaction, such as α-amylases, β-amylases, CGTases etc.

In one embodiment the concentration of starch in liquefaction mixture was about 15-40% (wt/wt), preferably about 20-30%.

In one embodiment the liquefaction was conducted at about 70-90° C. during about 0.5-10 hours, preferably about 1-5 hours. After liquefaction the steviol glycosides were added to the mixture and dissolved. The preferred ratio of steviol glycosides to starch (kg of steviol glycosides per 1 kg of starch) is about 0.5-1.5, preferably about 0.8-1.2.

In one embodiment a second portion of CGTase preparation is added and the transglucosylation reaction is conducted at about 65-70° C. for about 24-48 hours.

In one embodiment The reaction was stopped by heating at about 95° C. for about 15 minutes to inactivate the enzymes, and the solution was treated with activated carbon, to obtain decolorized reaction mixture. The amount of activated carbon was about 0.02-0.4 grams per gram of solids, preferably about 0.05-0.2 grams per gram of solids. Alternatively cartridges and/or columns packed with granulated activated carbon, may be used.

The decolorized reaction mixture was further concentrated by vacuum evaporator and dried by means of a spray dryer.

The GSG compositions of present invention can be used as sweeteners in various consumables including food and beverage products. Non-limiting examples of food and beverage products include carbonated soft drinks, ready to drink beverages, energy drinks, isotonic drinks, low-calorie drinks, zero-calorie drinks, sports drinks, cola flavored carbonated soft drinks, fruit flavored carbonated soft drinks, berry flavored carbonated soft drinks, flavored teas, fruit and vegetable juices, juice drinks, dairy drinks, yoghurt drinks, alcohol beverages, powdered beverages, bakery products, cookies, biscuits, baking mixes, cereals, confectioneries, candies, toffees, chewing gum, dairy products, flavored milk, yoghurts, flavored yoghurts, cultured milk, soy sauce and other soy base products, salad dressings, mayonnaise, vinegar, frozen-desserts, meat products, fish-meat products, bottled and canned foods, tabletop sweeteners, fruits and vegetables.

Additionally the compositions can be used in drug or pharmaceutical preparations and cosmetics, including but not limited to toothpaste, mouthwash, cough syrup, chewable tablets, lozenges, vitamin preparations, and the like.

The compositions can be used “as-is” or in combination with other sweeteners, flavors and food ingredients.

Non-limiting examples of sweeteners include sucrose, glyceraldehyde, dihydroxyacetone, erythrose, threose, erythrulose, arabinose, lyxose, ribose, xylose, ribulose, xylulose, allose, altrose, allulose, galactose, glucose, gulose, idose, mannose, talose, fructose, psicose, sorbose, tagatose, mannoheptulose, sedoheltulose, octolose, fucose, rhamnose, arabinose, turanose, sialose, inulin, inulooligosaccharides, fructooligosaccharides, high fructose corn syrup (HFCS), maltodextrin, coupling sugar, honey, erythritol, xylitol, mannitol, sorbitol, inositol, stevia, rebaudioside A, rebaudioside B, rebaudioside C, rebaudioside D, rebaudioside E, rebaudioside F, rebaudioside G, rebaudioside H, rebaudioside I, rebaudioside J, rebaudioside K, rebaudioside L, rebaudioside M, rebaudioside N, rebaudioside O, dulcoside A, dulcoside B, rubusoside, steviolbioside, stevioside, other steviol glycosides occurring in Stevia rebaudiana plant, biosynthetic steviol glycosides, glycosylated steviol glycosides, glucosylated steviol glycosides (GSGs), mogroside IV, mogroside V, mogroside VI, Luo han guo, siamenoside, other mogrosides occurring in Siraitia grosvenorii fruits, monatin and its salts, curculin, glycyrrhizic acid and its salts, thaumatin, monellin, mabinlin, brazzein, hernandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, and cyclocarioside I, sugar alcohols, sucralose, potassium acesulfame, acesulfame acid and salts thereof, aspartame, alitame, saccharin and salts thereof, neohesperidin dihydrochalcone, naringin dihydrochalcone, cyclamate, cyclamic acid and salts thereof, neotame, advantame, and combinations thereof.

Non-limiting examples of flavors include lemon, orange, fruity, banana, grape, pear, pineapple, bitter almond, lemon, lime, cola, cinnamon, sugar, cotton candy, vanilla flavors.

Non-limiting examples of other food ingredients include flavors, acidulants, organic and amino acids, coloring agents, polyols, fibers, bulking agents, modified starches, gums, texturizers, preservatives, antioxidants, emulsifiers, stabilisers, thickeners, gelling agents.

The following examples illustrate various embodiments of the invention. It will be understood that the invention is not limited to the materials, proportions, conditions and procedures set forth in the examples, which are only illustrative.

### Example 1

### Preparation of CGTase

A strain of Bacillus stearothermophilus St-88 was inoculated in 2,000 liters of sterilized culture medium containing 1.0% starch, 0.25% corn extract, 0.5% (NH₄)₂SO₄, and 0.2% CaCO₃ (pH 7.0-7.5) at 56° C. for 24 hrs with continuous aeration (2,000 L/min) and agitation (150 rpm). The obtained culture broth was filtered using Kerasep 0.1 μm ceramic membrane (Novasep, France) to separate the cells. The cell-free permeate was further concentrated 5-fold on Persep 10 kDa ultrafilters (Orelis, France).

### Example 2

### Preparation of Glucosyl Steviol Glycosides (GSG) Composition

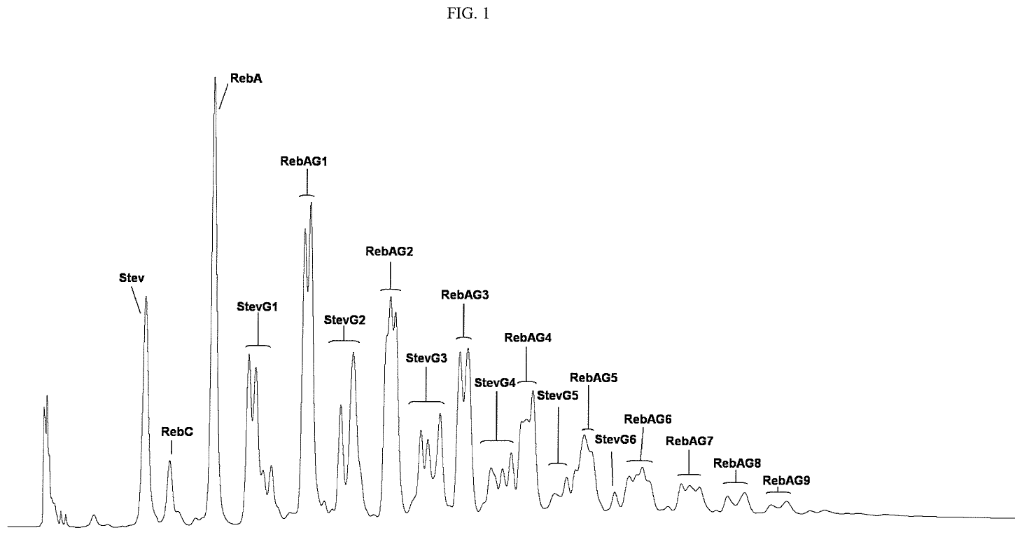

5,000 kg of tapioca starch was suspended in 13,600 L of water (pH 6) then 71 kg of CGTase, prepared according to Example 1, was added, and the liquefaction of starch was carried out at 83° C. for about 4 hours. Then 5,000 kg of stevia extract was added to liquefied starch and the mixture was heated in a heat exchanger to 105° C. until a homogeneous solution was obtained. 704 kg of CGTase, prepared according to Example 1, was added to the solution and the mixture was held at a temperature of 68° C. for 48 hours under continuous agitation. The mixture was passed through two 2 m³ columns packed with Norit GAC1240 PLUS granulated activated carbon (connected in in series) for decolorisation. The trace sediment in the filtrate (including residues the active carbon) was removed using a self de-sludging centrifuge (Alfa-Laval MPRX31456V-34C1/4271-6) and a dead end filter (Eaton, ACCUGAF, AGFE-51-RO2H0O-15L, pore size 1 μm). The decolorised solution was concentrated at 70° C. under vacuum, using NIRO thin falling film evaporator (running in MVR mode) until 60% (w/w) total solids content. The concentrated solution was dried into powder form using NIRO250 multistage spray dryer operating at an inlet temperature of 198° C. and outlet temperature of 77° C. 9,000 kg of glucosyl steviol glycosides (GSG) composition was obtained (FIG. 1).

We claim:

1. A sweetener composition comprising α-1,4-glucosyl derivatives of steviol glycosides.

2. A sweetener composition of claim 1 comprising at least one sweetener selected from the group consisting of sucrose, glyceraldehyde, dihydroxyacetone, erythrose, threose, erythrulose, arabinose, lyxose, ribose, xylose, ribulose, xylulose, allose, altrose, allulose, galactose, glucose, gulose, idose, mannose, talose, fructose, psicose, sorbose, tagatose, mannoheptulose, sedoheltulose, octolose, fucose, rhamnose, arabinose, turanose, sialose, inulin, inulooligosaccharides, fructooligosaccharides, high fructose corn syrup (HFCS), maltodextrin, coupling sugar, honey, erythritol, xylitol, mannitol, sorbitol, inositol, stevia, rebaudioside A, rebaudioside B, rebaudioside C, rebaudioside D, rebaudioside E, rebaudioside F, rebaudioside G, rebaudioside H, rebaudioside I, rebaudioside J, rebaudioside K, rebaudioside L, rebaudioside M, rebaudioside N, rebaudioside O, dulcoside A, dulcoside B, rubusoside, steviolbioside, stevioside, other steviol glycosides occurring in Stevia rebaudiana plant, biosynthetic steviol glycosides, glycosylated steviol glycosides, mogroside IV, mogroside V, mogroside VI, Luo han guo, siamenoside, other mogrosides occurring in Siraitia grosvenorii fruits, monatin and its salts, curculin, glycyrrhizic acid and its salts, thaumatin, monellin, mabinlin, brazzein, hernandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, and cyclocarioside I, sugar alcohols, sucralose, potassium acesulfame, acesulfame acid and salts thereof, aspartame, alitame, saccharin and salts thereof, neohesperidin dihydrochalcone, naringin dihydrochalcone, cyclamate, cyclamic acid and salts thereof, neotame, advantame, and combinations thereof.

3. A consumable comprising sweetener composition of claim 1, selected from the group including carbonated soft drinks, ready to drink beverages, energy drinks, isotonic drinks, low-calorie drinks, zero-calorie drinks, sports drinks, cola flavored carbonated soft drinks, fruit flavored carbonated soft drinks, berry flavored carbonated soft drinks, flavored teas, fruit and vegetable juices, juice drinks, dairy drinks, yoghurt drinks, alcohol beverages, powdered beverages, bakery products, cookies, biscuits, baking mixes, cereals, confectioneries, candies, chocolates, toffees, chewing gum, dairy products, flavored milk, yoghurts, flavored yoghurts, cultured milk, soy sauce and other soy base products, salad dressings, mayonnaise, vinegar, frozen-desserts, meat products, fish-meat products, bottled and canned foods, tabletop sweeteners, fruits and vegetables, drug or pharmaceutical preparations, cosmetics, toothpaste, mouthwash, cough syrup, chewable tablets, lozenges, vitamin preparations.

4. A consumable of claim 3, further comprising at least one food ingredient selected from the group consisting of: acidulants, organic and amino acids, coloring agents, bulking agents, modified starches, gums, texturizers, preservatives, antioxidants, emulsifiers, stabilisers, thickeners, gelling agents, and a combination thereof.

5. A method of sweetening a consumable by adding a composition of claim 1.
